(19) United States
(12) Patent Application Publication
(10) Pub. No.: US 2026/0331236 A1
(43) Pub. Date: Oct. 22, 2026

(54) SYNTHETIC PATIENT DATA GENERATION METHOD AND DEVICE UTILIZING LOCAL DIFFERENTIAL PRIVACY-BASED GENERATIVE ADVERSARIAL NETWORK

(71) Applicants: THE ASAN FOUNDATION, Seoul (KR); UNIVERSITY OF ULSAN FOUNDATION FOR INDUSTRY COOPERATION, Ulsan (KR)

(72) Inventors: Young-Hak KIM, Seoul (KR); Tae Joon JUN, Seoul (KR); Hansle GWON, Seoul (KR); Imjin AHN, Uijeongbu-si (KR); Heejun KANG, Seongnam-si (KR); Yunha KIM, Seoul (KR); Hyeram SEO, Seoul (KR); Hana CHO, Seoul (KR); Minkyoung KIM, Uijeongbu-si (KR); Jiye HAN, Seoul (KR); Gaeun KEE, Seoul (KR); Seohyun PARK, Seoul (KR); Heejung CHOI, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 19/111,363

(22) PCT Filed: Jul. 5, 2023
(86) PCT No.: PCT/KR2023/009488

§ 371 Date: Nov. 13, 2025

(30) Foreign Application Priority Data
Sep. 14, 2022 (KR) 10-2022-0115852

(51) Int. Cl. G16H 10/60; G06F 21/62; G06N 3/0475; G06N 3/094; G16H 50/70

(52) U.S. Cl. CPC G16H 10/60; G06F 21/6245; G06N 3/0475; G06N 3/094; G16H 50/70

(57) ABSTRACT

A method and a secure medical data generation electronic device that includes a memory in which computer-executable instructions are stored, and a processor which accesses the memory to execute the instructions. The instructions include generating perturbed patient data from patient data extracted from electronic medical records, generating perturbed pseudo data from pseudo data generated on the basis of a generator from a latent vector of a latent space, training a generative adversarial network-based model including a generator and a discriminator on the basis of the perturbed patient data and the perturbed pseudo data, and generating secured medical data by using the generator.

### TECHNICAL FIELD

The following description relates to a method and device for generating synthetic patient data utilizing a local differential privacy-based generative adversarial network (GAN).

### BACKGROUND ART

The medical field is one of the fields where artificial intelligence (AI) may be applied. It is because the introduction of AI to improve efficiency is essential since the number of patients is increasing, while medical resources are limited, and the amount and quality of medical data are increasing due to the development of medical devices. Numerous studies have shown that AI may make expert-level judgments when solving specific problems, and this is expanding its scope. However, there are still several issues that need to be solved for AI to be effectively applied in the medical field. The most important issue at present is the issue of security of private information of patient data, which is the most complex issue in handling medical data.

The above description has been possessed or acquired by the inventor(s) in the course of conceiving the present disclosure and is not necessarily an art publicly known before the present application is filed.

### DISCLOSURE OF THE INVENTION

### Technical Solutions

An electronic device for generating secured medical data according to an embodiment includes a memory configured to store computer-executable instructions, and a processor configured to execute the instructions by accessing the memory. The instructions are configured to, generate perturbed patient data from patient data extracted from electronic medical records (EMR), generate perturbed pseudo data from pseudo data generated based on a generator from a latent vector of a latent space, train a generative adversarial network (GAN)-based model including the generator and a discriminator based on the perturbed patient data and the perturbed pseudo data, and generate the secured medical data using the generator.

The processor may be configured to, when the extracted patient data is discrete data, generate the perturbed patient data including discrete data opposite to the patient data with a change probability based on a randomized response mechanism, and based on the discrete data, generate the perturbed pseudo data including discrete data opposite to the pseudo data with the change probability.

The processor may be configured to, when the extracted patient data is continuous data, generate noise data based on a Laplace mechanism, generate the perturbed patient data by adding the noise data to the patient data, and generate the perturbed pseudo data by adding the noise data to the pseudo data.

The processor may be configured to, based on a security adjustment level selected by a user, generate noise in a distribution corresponding to the security adjustment level, generate the perturbed patient data by adding the noise to the extracted patient data, and generate the perturbed pseudo data by adding the noise to the pseudo data.

The processor may be configured to generate the noise based on at least one of a randomized response mechanism, a Laplace mechanism, a Gaussian mechanism, or an exponential mechanism.

The processor may be configured to generate first noise and second noise based on at least two of the plurality of mechanisms, generate the perturbed patient data by adding the first noise to the extracted patient data, and generate the perturbed pseudo data by adding the second noise to the pseudo data.

The processor may be configured to obtain a discriminator score based on results of individually applying the discriminator to the perturbed pseudo data and the perturbed patient data, train the GAN-based model so that the generator generates the pseudo data similar to the patient data based on the discriminator score, train the GAN-based model so that the discriminator generates a result indicating true for the perturbed patient data based on the discriminator score, and train the GAN-based model so that the discriminator generates a result indicating false for the perturbed pseudo data based on the discriminator score.

The processor may be configured to obtain the discriminator score using an objective function of the discriminator based on a Wasserstein distance.

### BRIEF DESCRIPTION OF DRAWINGS

FIG. 1 is a diagram illustrating a membership inference attack.

FIG. 2 is a diagram illustrating a full black-box attack on a generator.

FIG. 3 is a diagram illustrating a partial black-box attack on a generator.

FIG. 4 is a diagram illustrating a discriminator-white-box attack on a discriminator.

FIG. 5 is a diagram illustrating a generative adversarial network (GAN)-based model for generating secured medical data according to an embodiment.

FIG. 6 is a diagram illustrating a method of training a GAN-based model according to an embodiment.

FIG. 7 is a flowchart illustrating a method of training a GAN according to an embodiment.

FIG. 8 is a diagram illustrating a method of applying noise to each patient data item according to an embodiment.

FIG. 9 is a diagram illustrating a randomized response mechanism according to an embodiment.

FIG. 10 is a diagram illustrating a Laplace mechanism according to an embodiment.

FIG. 11 is a diagram illustrating a method of generating pieces of perturbed data for training a GAN based on a security adjustment level according to an embodiment.

FIG. 12 is a diagram illustrating a method of generating pieces of perturbed data for training a GAN by applying a first mechanism and a second mechanism according to an embodiment.

FIG. 13 is a diagram illustrating a method of verifying utility of data according to an embodiment.

FIG. 14 is a diagram illustrating a device for generating secured medical data according to an embodiment.

### BEST MODE FOR CARRYING OUT THE INVENTION

The following detailed structural or functional description is provided as an example only and various alterations and modifications may be made to the embodiments. Accordingly, the embodiments are not construed as limited to the disclosure and should be understood to include all changes, equivalents, and replacements within the idea and the technical scope of the disclosure.

Although terms, such as first, second, and the like are used to describe various components, the components are not limited to the terms. These terms should be used only to distinguish one component from another component. For example, a first component may be referred to as a second component, or similarly, the second component may be referred to as the first component.

It should be noted that if it is described that one component is “connected,” “coupled,” or “joined” to another component, a third component may be “connected,” “coupled,” and “joined” between the first and second components, although the first component may be directly connected, coupled, or joined to the second component.

The singular forms “a,” “an,” and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

As used herein, “A or B,” “at least one of A and B,” “at least one of A or B,” “A, B or C,” “at least one of A, B and C,” and “at least one of A, B, or C,” each of which may include any one of the items listed together in the corresponding one of the phrases, or all possible combinations thereof.

Unless otherwise defined, all terms, including technical and scientific terms, used herein have the same meaning as commonly understood by one of ordinary skill in the art to which this disclosure pertains. Terms, such as those defined in commonly used dictionaries, should be construed to have meanings matching with contextual meanings in the relevant art, and are not to be construed to have an ideal or excessively formal meaning unless otherwise defined herein.

Hereinafter, embodiments will be described in detail with reference to the accompanying drawings. When describing the embodiments with reference to the accompanying drawings, like reference numerals refer to like elements and a repeated description related thereto will be omitted.

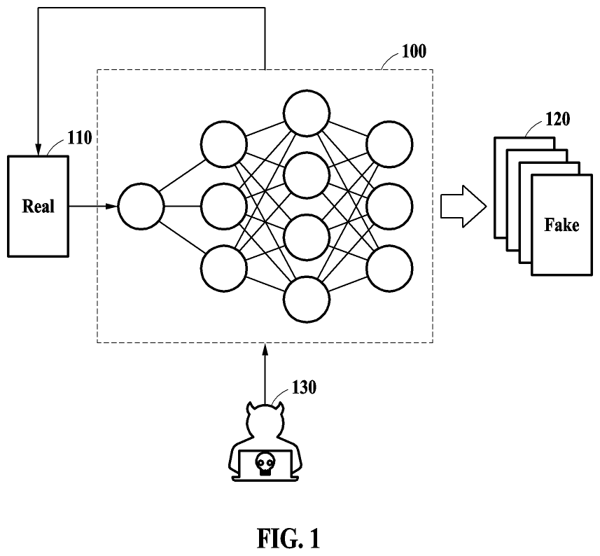

FIG. 1 is a diagram illustrating a membership inference attack.

An attacker 130 may obtain training data 110 used for training based on a trained machine learning model 100. FIG. 1 illustrates an example of an attack scenario by the attacker. For example, the attacker 130 may represent a person with expert knowledge about a system or programming within a computer without participating in the training process of the machine learning model 100. As will be described later, the machine learning model according to an embodiment may defend against attacks by the attacker described above.

The machine learning model 100 may be generated through machine learning. Learning algorithms may include, but are not limited to, for example, supervised learning, unsupervised learning, semi-supervised learning, or reinforcement learning. The machine learning model 100 may include a plurality of artificial neural network layers. An artificial neural network may include, for example, a deep neural network (DNN), a convolutional neural network (CNN), a recurrent neural network (RNN), a restricted Boltzmann machine (RBM), a deep belief network (DBN), and a bidirectional recurrent deep neural network (BRDNN), a deep Q-network, or a combination of two or more thereof, but is not limited thereto. For reference, in a case of supervised learning, the machine learning model 100 described above may be trained based on training data including pairs of the training data 110 and a training output 120 mapped to a corresponding training input. For example, the machine learning model 100 may be trained to output the training output 120 from the training data 110. The machine learning model 100 in training may generate a temporary output in response to the training data 110, and may be trained to minimize a loss between the temporary output and the training output 120. During the training process, a parameter (e.g., a connection weight between nodes and layers in a neural network) of the machine learning model 100 may be updated based on the loss. Such learning may be performed by, for example, an electronic device in which the machine learning model 100 is performed, or performed via a separate server. The machine learning model 100 that is trained may be stored in a memory of an electronic device.

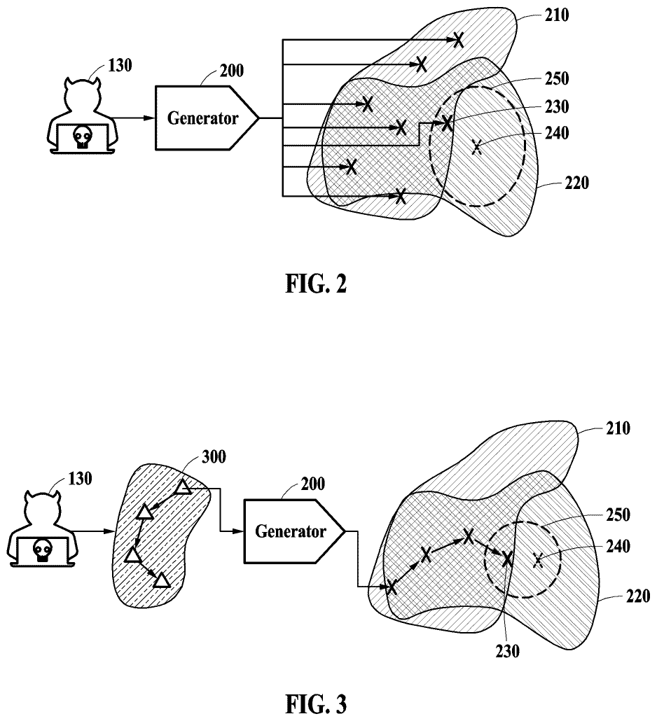

The attacker 130 may obtain the training data 110 based on a random output obtained by applying random data to the machine learning model 100. The attack scenario in which the attacker 130 obtains the training data 110 will be described below with reference to FIGS. 2 to 4.

FIG. 2 is a diagram illustrating a full black-box attack on a generator.

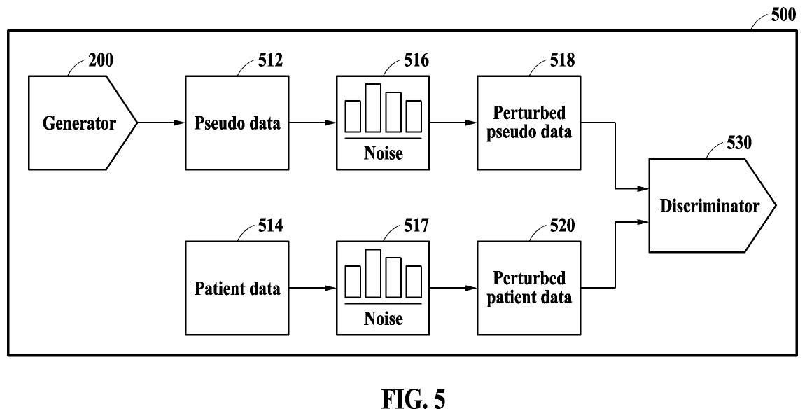

The attacker 130 may generate pieces of randomized data in a generator 200. For example, the attacker 130 may obtain restored data 230 similar to target data 240 based on the generated randomized data. The restored data 230 may represent data most similar to the target data 240 among the pieces of randomized data generated by the attacker 130. The target data 240 may represent training data used for the training of the generator 200. A generated area 210 in FIG. 2 may represent an area of the pieces of randomized data generated by the attacker 130 through the generator 200. The randomized data may represent pseudo data which will be described below with reference to FIG. 5. An actual area 220 may represent an area of training data used for the training of the generator 200. Specifically, when distance data of the restored data 230 and the target data 240 is within a threshold distance 250 of the target data 240, the attacker 130 may evaluate the restored data 230 as training data used for the training of the generator 200.

The distance data may be calculated by the following equation.

$$R = {arg min}_{\hat{X}} L ( X , {X}_{15}^{\hat{}} ) \qquad [ Equation 1 ]$$

Here, X may represent the target data 240, and \hat{X} may represent data belonging to the generated area 210. Also, L may represent a distance function. For example, the distance function may use a Euclidean distance.

FIG. 3 is a diagram illustrating a partial black-box attack on a generator.

The attacker 130 may generate pieces of randomized data in the generator 200 based on a latent vector 300. The latent vector 300 may represent input data of the generator 200 for generating the pieces of randomized data in the generator 200. For example, the attacker 130 may obtain the restored data 230 similar to the target data 240 based on the generated randomized data. For reference, unlike the full black-box attack, the attacker 130 may obtain the restored data 230 by modifying the latent vector 300 in a partial black box attack. Also, the attacker 130 may obtain an optimal latent vector based on the latent vector 300 and the restored data 230.

The optimal latent vector may be calculated by the following equation.

$${z}^{*} = {arg min}_{z} L ( X , G ( z ) ) \qquad [ Equation 2 ]$$

Here, G(z) may represent randomized data generated by the generator 200 based on the latent vector 300.

The restored data 230 may represent data most similar to the target data 240 among the pieces of randomized data generated by the attacker 130. When distance data of the restored data 230 and the target data 240 is within a threshold distance 250 of the target data 240, the attacker 130 may evaluate the restored data 230 as training data used for the training of the generator 200.

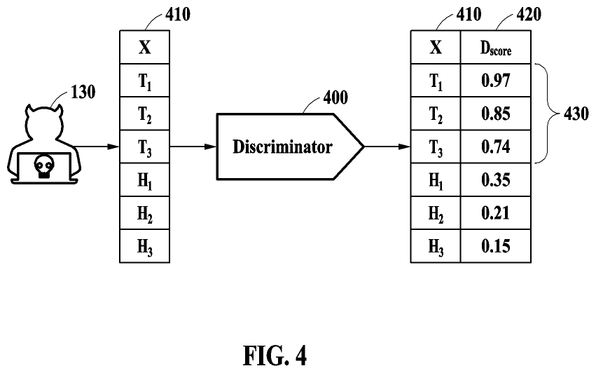

FIG. 4 is a diagram illustrating a discriminator-white-box attack on a discriminator.

The attacker 130 may obtain data used for the training through a discriminator score 420 of a discriminator 400. For example, the attacker 130 may obtain pieces of inference data 430 including discriminator scores exceeding a threshold value among the discriminator score 420 of a plurality of pieces of data 410. The plurality of pieces of data 410 may include at least one of data used for the training of the discriminator 400 or data not used for the training of the discriminator 400. The discriminator 400 may be trained to output a higher discriminator score 420 for the data used for the training of the discriminator 400. In contrast, the discriminator 400 may be trained to output a lower discriminator score 420 for the data not used for the training of the discriminator 400.

Here, the attacker 130 may obtain the pieces of inference data 430 through the characteristics of the discriminator 400 described above.

FIG. 5 is a diagram illustrating a generative adversarial network (GAN)-based model for generating secured medical data according to an embodiment.

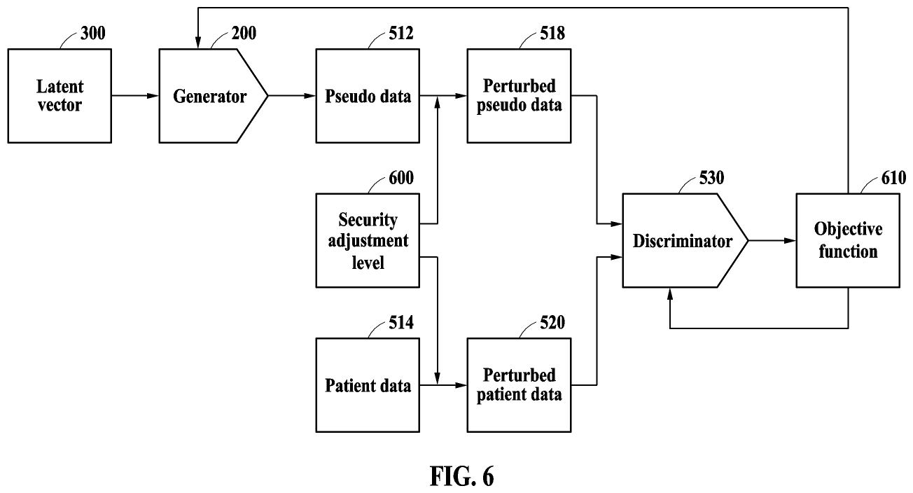

The electronic device according to an embodiment may apply pieces of perturbed data to a GAN-based model 500. For example, the GAN-based model 500 may include the generator 200 and a discriminator 530. The generator 200 may represent a machine learning model that receives one random vector included in a latent space and outputs pseudo data 512 which will be described below with reference to FIG. 6. The generator 200 may generate the pseudo data 512 so that the discriminator 530 may not discriminate between perturbed pseudo data 518 and perturbed patient data 520. Patient data 514 may represent data including private information about a plurality of patients. For example, patient data may include at least one of gender, age, height, systolic blood pressure, diastolic blood pressure, body mass index (BMI), or procedure experience of patients. The pseudo data 512 may represent fake data generated by the generator 200. The pseudo data 512 may include at least one of discrete data or continuous data depending on the type of the patient data 514. For example, the discrete data may represent data having a discrete value among the patient data 514. The continuous data may represent data having a continuous real value among the patient data 514. When the patient data 514 includes the discrete data, the pseudo data 512 may also include the discrete data, and when the patient data 514 includes the continuous data, the pseudo data 512 may also include the continuous data.

The pieces of perturbed data may represent data that is different from original data due to noise being applied to the original data. Here, the perturbed pseudo data 518 and the perturbed patient data 520 are described as examples of perturbed data. The perturbed pseudo data 518 may be generated by applying noise 516 to the pseudo data 512. The perturbed patient data 520 may be generated by applying noise 517 to the patient data 514. The generator 200 may be trained so that the discriminator 530 may not distinguish between the perturbed pseudo data 518 and the perturbed patient data 520. A method of training the GAN-based model 500 will be described below with reference to FIG. 6.

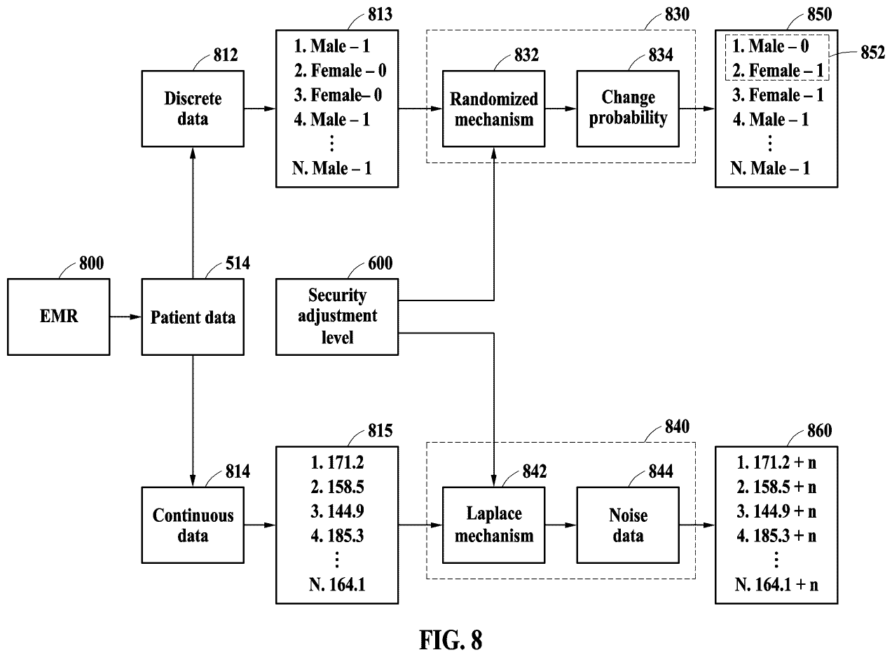

The noise 516 and 517 may represent at least one of noise data representing a mathematically designed random real value based on differential privacy or data to which a change probability is applied. The differential privacy may represent a methodology for quantitatively modeling privacy and measuring a degree of privacy protection. The method of obtaining the noise 516 and 517 through the differential privacy will be described below with reference to FIGS. 8 to 10.

FIG. 6 is a diagram illustrating a method of training a GAN-based model according to an embodiment.

An electronic device according to an embodiment may train a GAN-based model (e.g., the GAN-based model 500 of FIG. 5) based on a security adjustment level 600. For example, the security adjustment level 600 is a parameter representing the intensity of noise (e.g., the noise 516 and 517 of FIG. 5) and may be input by a user.

For example, when the increased security adjustment level 600 is received from the user, the electronic device may obtain pieces of data perturbed by increased noise intensity. The electronic device may apply, to the data, noise having the noise intensity corresponding to the increased security adjustment level 600 input by the user. Through the noise with the increased intensity, it may be possible to improve the security of the data (e.g., the pseudo data 512 or the patient data 514). As another example, the electronic device may generate pieces of perturbed data based on the security adjustment level 600 that represents noise with reduced intensity, in order to increase the utility of the data.

Specifically, the electronic device may apply the security adjustment level 600 that satisfies the following equation to the pseudo data 512 and the patient data 514.

$$\vert log ( \frac{P r [ A ( D ) ∈ S ]}{P r [ A ( {D}^{′} ) ∈ S ]} ) \vert \leq ε \qquad [ Equation 3 ]$$

Here, ε may represent the security adjustment level 600, and D and D′ may represent a set of data that differ only in one data point. For example, when the set of data is a set of patient data 514, D and D′ may represent a set of data that differs only in one piece of patient data among a plurality of pieces of patient data. Also, A may represent a differential privacy mechanism, and S′ may represent a subset of the results of applying the differential privacy mechanism to the data set described above. As a result, the electronic device may generate the perturbed pseudo data 518 and the perturbed patient data 520 by applying the security adjustment level 600 satisfying Equation 3 to the pseudo data 512 and the patient data 514. A method of training the GAN-based model based on the pieces of perturbed data will be described below.

The electronic device according to an embodiment may train the generator 200 and the discriminator 530 through an objective function 610 of the discriminator 530.

The objective function 610 of the discriminator may be represented by the following equation.

$${maximize}_{D} ( {E}_{x \sim {p}_{x}} [ log ( D ( x ) ) ] + {E}_{z \sim {p}_{z}} [ log ( 1 - D ( G ( z ) ) ] ) \qquad [ Equation 4 ]$$

Here, D(x) may represent a discriminator score obtained by applying the perturbed patient data 520 to the discriminator 530, and D(G(z)) may represent a discriminator score obtained by applying the perturbed pseudo data 518 to the discriminator 530. In addition, E_{x~Px}[log (D(x))] may represent an expected value of based on sample patient data following a distribution of the perturbed patient log (D(x)) data 520. E_{z~Pz}[log (1−D(G(z))] may represent an expected value of log (1-D(G(z)) based on sample pseudo data following a distribution of the perturbed pseudo data 518. The discriminator score may represent a score that includes whether the data applied to the discriminator 530 is real data (e.g., the perturbed patient data 520) or fake data (e.g., the perturbed pseudo data 518). For example, the discriminator score may include a score close to 1 when the data input to the discriminator 530 is real data. Specifically, the electronic device may obtain the discriminator 530 whose discriminative ability improves as training progresses, and the generator 200 which is sophisticated to fool the discriminative ability of the discriminator 530.

The electronic device according to an embodiment may obtain the discriminator score through the objective function of the discriminator based on a Wasserstein distance, in order to obtain the discriminator score. The Wasserstein distance may represent a distance when the expected value of the distance is the smallest by measuring the correlation of two probability distributions.

The Wasserstein distance may be calculated by the following equation.

$$W ( {P}_{r} , {P}_{Θ} ) = {sup}_{{‖ f ‖}_{L \leq 1}} \cdot {E}_{x \sim {P}_{r}} [ f ( x ) ] - {E}_{x \sim {P}_{Θ}} [ f ( x ) ] \qquad [ Equation 5 ]$$

Here, X may represent input data (e.g., at least one of patient data or pseudo data), and f(x) may represent the output of the discriminator (e.g., the discriminator score). In addition, Pᵣ may represent the distribution of patient data, P_{θ} may represent the distribution of pseudo data, and

$${sup}_{{‖ f ‖}_{L \leq 1}}$$

may represent a supremum value where an absolute value of the slope does not exceed 1.

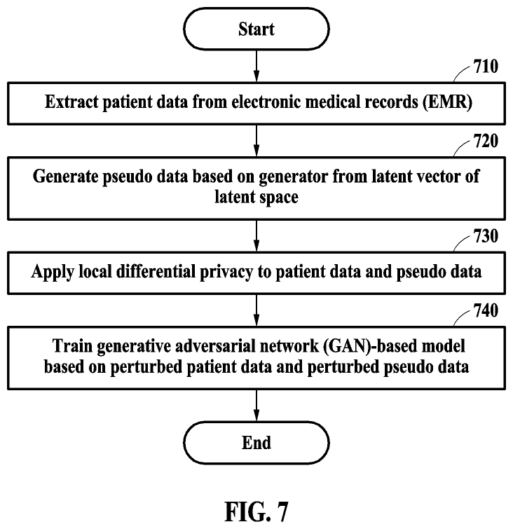

FIG. 7 is a flowchart illustrating a method of training a GAN according to an embodiment.

In operation 710, the electronic device may extract patient data from electronic medical records (EMR). For example, the EMR may refer to computerized information recorded so that data about patients using a medical system may be managed efficiently and uniformly. Specifically, the EMR may include at least one of discrete data or continuous data which will be described below with reference to FIGS. 8 to 10.

In operation 720, the electronic device may generate pseudo data (e.g., the pseudo data 512 of FIG. 5) based on a generator (e.g., the generator 200 of FIG. 2) from a latent vector of a latent space. The pseudo data, unlike the patient data, may include data that is simply output by the generator. Specifically, the pseudo data may not be completely dependent on institutions because the pseudo data does not include private information about patients, and may include data that may be utilized relatively freely under multiple legal regulations.

In operation 730, the electronic device may apply local differential privacy to the patient data and the pseudo data. For example, the electronic device may apply a change probability generated through the local differential privacy to the patient data and the pseudo data based on the patient data being binary data. The electronic device may add noise data generated through the local differential privacy to the patient data and the pseudo data based on the patient data being continuous data. A method of applying the local differential privacy to the patient data and the pseudo data will be described below with reference to FIGS. 8 to 10.

In operation 740, the electronic device may train a GAN-based model (e.g., the GAN-based model 500 of FIG. 5) based on perturbed patient data (e.g., the perturbed patient data 520 of FIG. 5) and perturbed pseudo data (e.g., the perturbed pseudo data 518 of FIG. 5). Specifically, the electronic device may train the GAN-based model based on the perturbed patient data and the perturbed pseudo-data, thereby preventing the private information included in the patient data from being leaked by an external attacker.

FIG. 8 is a diagram illustrating a method of applying noise to each patient data item according to an embodiment.

An electronic device according to an embodiment may apply noise to the patient data 514 based on the security adjustment level 600. For example, the patient data 514 may be extracted from an EMR 800. The patient data 514 may include at least one of discrete data 812 or continuous data 814.

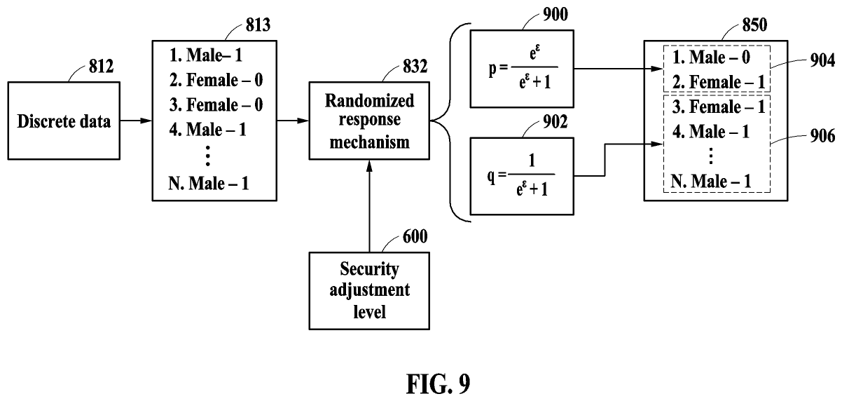

The electronic device according to an embodiment may obtain perturbed discrete data 850 by applying first noise 830 to the discrete data 812. For example, the discrete data 812 may include information expressed as discrete values. Specifically, the discrete data 812 may include pieces of data expressed as binary values. In this specification, for the convenience of description, a gender binary value 813 corresponding to the patient's gender will be mainly described as an example of the discrete data 812, but embodiments are not limited thereto. The gender binary value 813 may include data represented as a binary value of 1 for a male patient and 0 for a female patient. The first noise 830 may include a randomized response mechanism 832 and a change probability 834. For example, the randomized response mechanism 832 is a representative mechanism for discrete data among the mechanisms of local differential privacy to be described below, and may represent a mechanism for generating a binary value converted to another binary value among binary values corresponding to a change probability 534 based on the gender binary value 813. Here, the electronic device may obtain the change probability 834 by applying the security adjustment level 600 to the randomized response mechanism 832. The electronic device may obtain the perturbed discrete data 850 by applying the obtained change probability 834 to the gender binary value 813. The perturbed discrete data 850 may include a binary value 852 that is converted to another binary value among the original data (e.g., the gender binary value 813) by the change probability. The randomized response mechanism 832 will be described below with reference to FIG. 9.

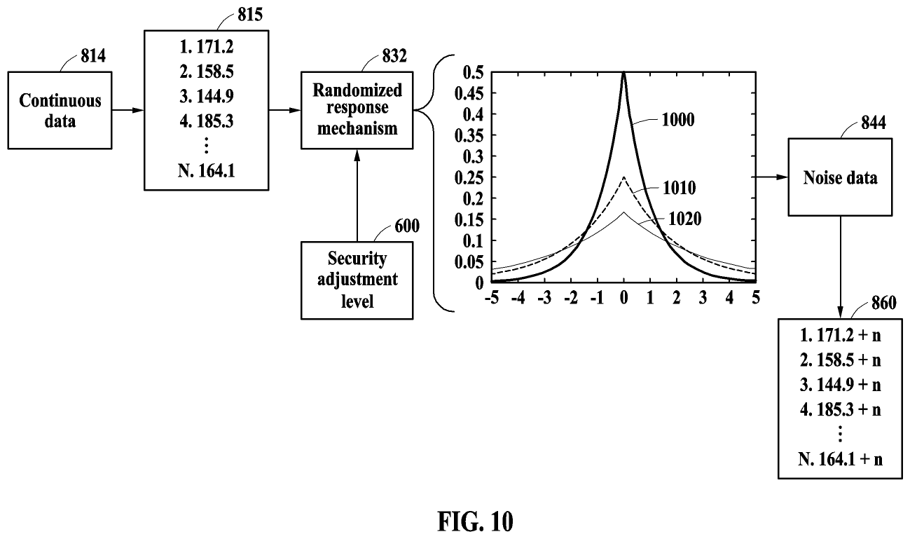

The electronic device according to an embodiment may obtain perturbed continuous data 860 by applying second noise 840 to the continuous data 814. For example, the continuous data 814 may include information expressed as continuous values. In this specification, for the convenience of description, patient's height information 815 will be mainly described as an example of the continuous data 814, but embodiments are not limited thereto. The second noise 840 may include a Laplace mechanism 842 and noise data 844. For example, the Laplace mechanism 842 is a representative mechanism for continuous data among the mechanisms of local differential privacy to be described below, and may represent a mechanism for adding the noise data 844 to the continuous data. Here, the electronic device may obtain the noise data 844 by applying the security adjustment level 600 to the Laplace mechanism 842. The electronic device may obtain the perturbed continuous data 860 by adding the obtained noise data 844 to the patient's height information 815. The perturbed continuous data 860 may include perturbed patient's height information with the noise data 844 added. The Laplace mechanism 842 will be described below with reference to FIG. 10.

FIG. 9 is a diagram illustrating a randomized response mechanism according to an embodiment.

An electronic device according to an embodiment may obtain the perturbed discrete data 850 by applying the randomized response mechanism 832 to the discrete data 812. The discrete data 812 may include the gender binary value 813. For example, the electronic device may obtain a change probability 900 and a maintenance probability 902 by applying the security adjustment level 600 based on at least one of a user input or a predetermined value to the randomized response mechanism 832. The electronic device may obtain the perturbed discrete data 850 based on the change probability 900 and the maintenance probability 902.

The change probability 900 may be calculated by the following equation.

$$p = \frac{{e}^{ε}}{{e}^{ε} + 1} \qquad [ Equation 6 ]$$

Here, ε may represent the security adjustment level 600, e may represent the base of the natural logarithm, and p may represent the change probability 900.

The perturbed discrete data 850 may include a result of applying the randomized response mechanism 832 to the gender binary value 813, which is an example of the discrete data 812. For example, the perturbed discrete data 850 may include changed discrete data 904 and maintained discrete data 906. The changed discrete data 904 may represent discrete data changed from the gender binary value 813 by the change probability 900. The maintained discrete data 906 may represent discrete data maintained from the gender binary value 813 by the maintenance probability 902. As a result, the electronic device may train a GAN-based model (e.g., the model 500 of FIG. 5) using the perturbed discrete data 850 as the perturbed pseudo data (e.g., the perturbed pseudo data 518 of FIG. 5) or the perturbed patient data (e.g., the perturbed patient data 520 of FIG. 5).

FIG. 10 is a diagram illustrating a Laplace mechanism according to an embodiment.

An electronic device according to an embodiment may obtain the perturbed continuous data 860 by applying the Laplace mechanism 842 to the continuous data 814. The continuous data 814 may include the patient's height information 815. For example, the electronic device may obtain Laplace distributions by applying the security adjustment level 600 based on at least one of a user input or a predetermined value to the Laplace mechanism 842. In this specification, for the convenience of description, a plurality of Laplace distributions will be described as, for example, three Laplace distributions, but embodiments are not limited thereto. For example, the electronic device may obtain a first distribution 1000 to a third distribution 1020 based on the plurality of security adjustment levels 600. The first distribution 1000 may include values having a smaller security adjustment level than a second distribution 1010 and the third distribution 1020.

The electronic device according to an embodiment may obtain the perturbed continuous data 860 by adding the noise data 844 to the continuous data 814. The perturbed continuous data 860 may include a result of applying the Laplace mechanism 842 to the patient's height information 815, which is an example of the continuous data 814. For example, the perturbed continuous data 860 may include data in which the noise data 844 is added to the patient's height information 815. As a result, the electronic device may train a GAN-based model (e.g., the model 500 of FIG. 5) using the perturbed continuous data 860 as the perturbed pseudo data (e.g., the perturbed pseudo data 518 of FIG. 5) or the perturbed patient data (e.g., the perturbed patient data 520 of FIG. 5).

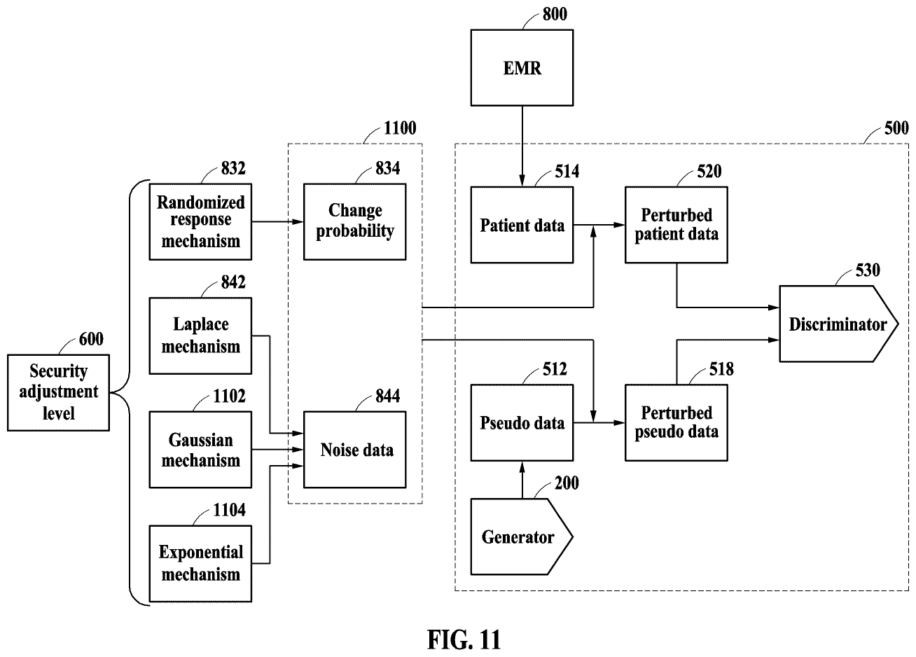

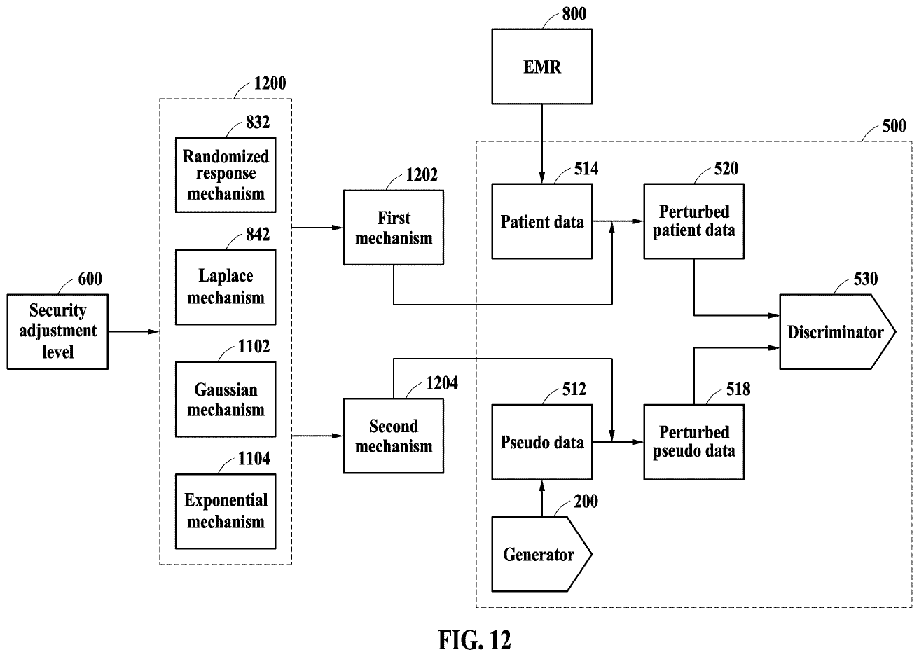

FIG. 11 is a diagram illustrating a method of generating pieces of perturbed data for training a GAN based on a security adjustment level according to an embodiment.

An electronic device according to an embodiment may train a GAN-based model based on the security adjustment level 600. For example, the electronic device may train the GAN-based model by applying the security adjustment level 600 to one of a plurality of mechanisms. The plurality of mechanisms may include at least one of the randomized response mechanism 832, the Laplace mechanism 842, a Gaussian mechanism 1102, or an exponential mechanism 1104.

The electronic device according to an embodiment may generate the perturbed pseudo data 518 by applying a noise parameter 1100 to the pseudo data 512. The electronic device may generate the perturbed patient data 520 by applying the noise parameter 1100 to the patient data 514. The noise parameter 1100 may represent a parameter related to the noise intensity added to data based on a mechanism selected according to the type of the patient data 514. Specifically, the noise parameter 1100 may include at least one of the change probability 834 or the noise data 844. For example, based on the patient data 514 being discrete data (e.g., the discrete data 812 of FIG. 8), the electronic device may generate the change probability 834 by the randomized response mechanism 832.

The electronic device according to an embodiment may generate a plurality of pieces of noise data 844 through the Laplace mechanism 842. For example, the electronic device may obtain a first distribution (e.g., the first distribution 1000 of FIG. 10) by the Laplace mechanism 842 based on the security adjustment level 600. The electronic device may generate the plurality of pieces of noise data 844 in the first distribution. The electronic device may apply two of the plurality of pieces of noise data 844 to the patient data 514 and the pseudo data 512, respectively.

FIG. 12 is a diagram illustrating a method of generating pieces of perturbed data for training a GAN by applying a first mechanism and a second mechanism according to an embodiment.

An electronic device according to an embodiment may generate perturbed data by applying a first mechanism 1202 and a second mechanism 1204. For example, the electronic device may select at least two different mechanisms from among a plurality of mechanisms 1200. Specifically, when the patient data 514 is discrete data (e.g., the discrete data 812 of FIG. 8), both the first mechanism 1202 and the second mechanism 1204 may represent the randomized response mechanism 832. When the patient data 514 is continuous data (e.g., the continuous data 814 of FIG. 8), the first mechanism 1202 and the second mechanism 1204 may represent at least two different mechanisms among the Laplace mechanism 842, the Gaussian mechanism 1102, or the exponential mechanism 1104.

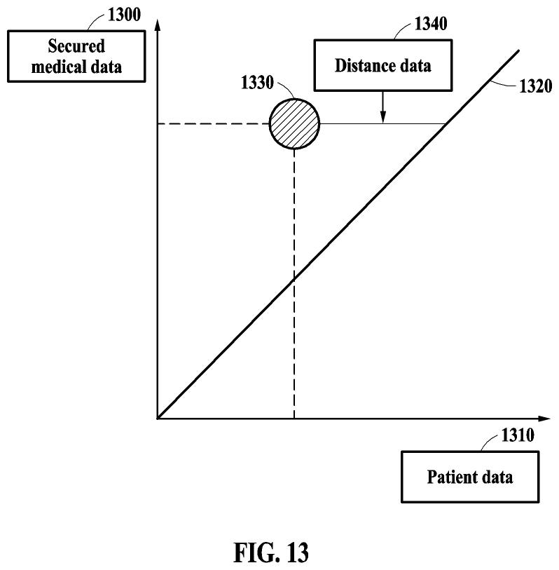

FIG. 13 is a diagram illustrating a method of verifying utility of data according to an embodiment.

The utility of data according to an embodiment may be evaluated by at least one of a scatter plot 1330 or a correlation. The utility of data may represent a degree of similarity between secured medical data 1300 and patient data 1310. The scatter plot 1330 may represent a point where the patient data 1310 on the x-axis and the secured medical data 1300 on the y-axis meet. For example, the scatter plot 1330 may include a point represented as a dot on a coordinate plane by calculating a score according to the characteristics of a data item. The score may be obtained based on at least one of dimension wise statistics (DWS), dimension wise average (DWA), or dimension wise prediction (DWP) according to information to be identified or characteristics of the data item. The secured medical data 1300 may represent data generated by a generator (e.g., the generator 200 of FIG. 2) of the trained GAN-based model (e.g., the GAN-based model 500 of FIG. 5).

The utility of data according to an embodiment may include distance data 1340. The distance data 1340 may represent an indicator of the evaluated utility of the data. As shown in FIG. 13, the utility of the secured medical data 1300 may be obtained based on the scatter plot 1330 and a proportional utility index 1320. The proportional utility index 1320 may indicate that the patient data 1310 and the secured medical data 1300 are identical. As a result, the electronic device may use the pieces of perturbed data (e.g., the perturbed pseudo data 518 or the perturbed patient data 520 of FIG. 5) to train the GAN-based model for the security of the patient data 1310, and may obtain the utility of the data through the secured medical data 1300 generated by the generator which is trained similarly to the actual patient data 1310.

The utility of data according to an embodiment may be evaluated by the correlation of the secured medical data 1300 and the patient data 1310.

The correlation may be calculated by the following equation.

Corr=mean(|CMᵣₑₐₗ−CM_{fake}|) [Equation 7]

Here, CMᵣₑₐₗ may represent a correlation matrix of the patient data 1310, and CM_{fake} may represent a correlation matrix of the secured medical data 1300.

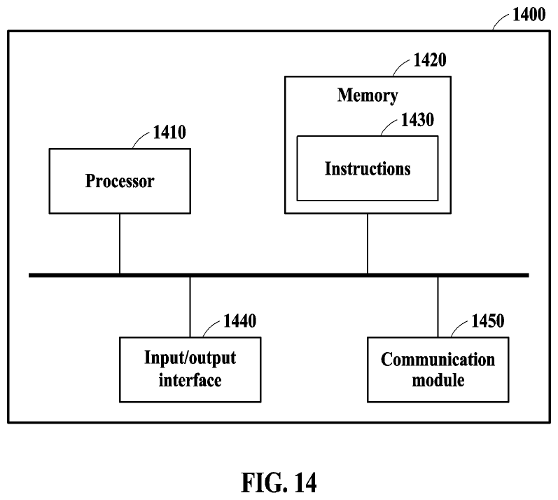

FIG. 14 is a diagram illustrating a device for generating secured medical data according to an embodiment.

An electronic device 1400 according to an embodiment may include a processor 1410, a memory 1420, an input/output interface 1440, and a communication module 1450.

The processor 1410 may train a GAN-based model (e.g., the GAN-based model 500 of FIG. 5) including a generator (e.g., the generator 200 of FIG. 5) and a discriminator (e.g., the discriminator 530 of FIG. 5) based on perturbed patient data (e.g., the perturbed patient data 520 of FIG. 5) and perturbed pseudo data (e.g., the perturbed pseudo data 518 of FIG. 5). The processor 1410 may execute software to control at least another component (e.g., a hardware or software component) connected to the processor 1410. The processor 1410 may also perform a variety of data processing or computation.

The memory 1420 may include computer-executable instructions 1430. The memory 1420 may temporarily and/or permanently store a variety of data and/or information required to train a GAN-based model. For example, the memory 1420 may store at least one of perturbed patient data, perturbed pseudo data, or a GAN-based model.

The input/output interface 1440 may include an input device and an output device. The input device may receive an input from a user through a tactile input, a video input, an audio input, or a touch input. The input device may include, for example, a keyboard, a mouse, a touchscreen, a microphone, and other devices that may detect an input from a user and transmit the detected input to the electronic device 1400. The output device may provide a user with an output of the electronic device 1400 through a visual channel, an auditory channel, or a tactile channel. The output device may include, for example, a display, a touchscreen, a speaker, a vibration generator, or any other device configured to provide an output to the user. The communication circuit 1450 may communicate with an external device through a wired or wireless network.

The embodiments described herein may be implemented using a hardware component, a software component and/or a combination thereof. A processing device may be implemented using one or more general-purpose or special-purpose computers, such as, for example, a processor, a controller, an arithmetic logic unit (ALU), a digital signal processor (DSP), a microcomputer, a field programmable gate array (FPGA), a programmable logic unit (PLU), a microprocessor or any other device capable of responding to and executing instructions in a defined manner. The processing device may run an operating system (OS) and one or more software applications that run on the OS. The processing device also may access, store, manipulate, process, and generate data in response to execution of the software. For purpose of simplicity, the description of a processing device is used as singular; however, one skilled in the art will appreciate that a processing device may include multiple processing elements and/or multiple types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software may also be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter.

The above-described hardware devices may be configured to act as one or more software modules in order to perform the operations of the above-described embodiments, or vice versa.

As described above, although the embodiments have been described with reference to the limited drawings, a person skilled in the art may apply various technical modifications and variations based thereon. For example, suitable results may be achieved if the described techniques are performed in a different order and/or if components in a described system, architecture, device, or circuit are combined in a different manner and/or replaced or supplemented by other components or their equivalents.

Therefore, other implementations, other embodiments, and equivalents to the claims are also within the scope of the following claims.

1. A method of generating secured medical data performed by a processor, the method comprising:
generating perturbed patient data from patient data extracted from electronic medical records (EMR);
generating perturbed pseudo data from pseudo data generated based on a generator from a latent vector of a latent space;
training a generative adversarial network (GAN)-based model comprising the generator and a discriminator based on the perturbed patient data and the perturbed pseudo data; and
generating the secured medical data using the generator.

2. The method of claim 1, further comprising:
when the extracted patient data is discrete data, generating the perturbed patient data comprising discrete data opposite to the patient data with a change probability based on a randomized response mechanism; and
based on the discrete data, generating the perturbed pseudo data comprising discrete data opposite to the pseudo data with the change probability.

3. The method of claim 1, further comprising:
when the extracted patient data is continuous data, generating noise data based on a Laplace mechanism;
generating the perturbed patient data by adding the noise data to the patient data; and
generating the perturbed pseudo data by adding the noise data to the pseudo data.

4. The method of claim 1, further comprising:
based on a security adjustment level selected by a user, generating noise in a distribution corresponding to the security adjustment level;
generating the perturbed patient data by adding the noise to the extracted patient data; and
generating the perturbed pseudo data by adding the noise to the pseudo data.

5. The method of claim 4, wherein the generating of the noise comprises generating the noise based on at least one of a randomized response mechanism, a Laplace mechanism, a Gaussian mechanism, or an exponential mechanism.

6. The method of claim 5, further comprising:
generating first noise and second noise based on at least two of the plurality of mechanisms;
generating the perturbed patient data by adding the first noise to the extracted patient data; and
generating the perturbed pseudo data by adding the second noise to the pseudo data.

7. The method of claim 1, wherein the training of the GAN-based model comprises:
obtaining a discriminator score based on results of individually applying the discriminator to the perturbed pseudo data and the perturbed patient data;
training the GAN-based model so that the generator generates the pseudo data similar to the patient data based on the discriminator score;
training the GAN-based model so that the discriminator generates a result indicating true for the perturbed patient data based on the discriminator score; and
training the GAN-based model so that the discriminator generates a result indicating false for the perturbed pseudo data based on the discriminator score.

8. The method of claim 7, wherein the obtaining of the discriminator score comprises obtaining the discriminator score using an objective function of the discriminator based on a Wasserstein distance.

9. A non-transitory computer-readable storage medium storing instructions that, when executed by a processor, cause the processor to perform the method of claim 1.

10. An electronic device for generating secured medical data, the electronic device comprising:
a memory configured to store computer-executable instructions; and
a processor configured to execute the instructions by accessing the memory,
wherein the instructions are configured to,
generate perturbed patient data from patient data extracted from electronic medical records (EMR),
generate perturbed pseudo data from pseudo data generated based on a generator from a latent vector of a latent space,
train a generative adversarial network (GAN)-based model comprising the generator and a discriminator based on the perturbed patient data and the perturbed pseudo data, and
generate the secured medical data using the generator.

11. The electronic device of claim 10, wherein the processor is configured to,
when the extracted patient data is discrete data, generate the perturbed patient data comprising discrete data opposite to the patient data with a change probability based on a randomized response mechanism, and
based on the discrete data, generate the perturbed pseudo data comprising discrete data opposite to the pseudo data with the change probability.

12. The electronic device of claim 10, wherein the processor is configured to,
when the extracted patient data is continuous data, generate noise data based on a Laplace mechanism,
generate the perturbed patient data by adding the noise data to the patient data, and
generate the perturbed pseudo data by adding the noise data to the pseudo data.

13. The electronic device of claim 10, wherein the processor is configured to,
based on a security adjustment level selected by a user, generate noise in a distribution corresponding to the security adjustment level,
generate the perturbed patient data by adding the noise to the extracted patient data, and
generate the perturbed pseudo data by adding the noise to the pseudo data.

14. The electronic device of claim 13, wherein the processor is configured to generate the noise based on at least one of a randomized response mechanism, a Laplace mechanism, a Gaussian mechanism, or an exponential mechanism.

15. The electronic device of claim 14, wherein the processor is configured to,
generate first noise and second noise based on at least two of the plurality of mechanisms,
generate the perturbed patient data by adding the first noise to the extracted patient data, and
generate the perturbed pseudo data by adding the second noise to the pseudo data.

16. The electronic device of claim 10, wherein the processor is configured to,
obtain a discriminator score based on results of individually applying the discriminator to the perturbed pseudo data and the perturbed patient data,
train the GAN-based model so that the generator generates the pseudo data similar to the patient data based on the discriminator score,
train the GAN-based model so that the discriminator generates a result indicating true for the perturbed patient data based on the discriminator score, and
train the GAN-based model so that the discriminator generates a result indicating false for the perturbed pseudo data based on the discriminator score.

17. The electronic device of claim 16, wherein the processor is configured to obtain the discriminator score using an objective function of the discriminator based on a Wasserstein distance.
